(19) United States
(12) Patent Application Publication
(10) Pub. No.: US 2009/0233997 A1
(43) Pub. Date: Sep. 17, 2009

(54) COMPOSITION FOR PROMOTING SYNTHESIS OF COLLAGEN, AND COMPOSITION FOR EXTERNAL PREPARATION FOR SKIN COMPRISING THE SAME

(75) Inventors: Mu-Hyun JIN, Daejeon-City (KR); Sang-Hwa Lee, Daejeon-City (KR); Yang-Mi Park, Daejeon-City (KR); Sang-Jin Kang, Daejeon-City (KR)

Correspondence Address: LADAS & PARRY, 5670 WILSHIRE BOULEVARD, SUITE 2100, LOS ANGELES, CA, 90036-5679, US

(73) Assignee: LG HOUSEHOLD & HEALTH CARE, LTD., Seoul (KR)

(21) Appl. No.: 12/405,964

(22) Filed: Mar. 17, 2009

Related U.S. Application Data
(62) Division of application No. 10/575,974, filed on Aug. 31, 2006.

(30) Foreign Application Priority Data
Oct. 17, 2003 (KR) 10-2003-0072539
Oct. 17, 2003 (KR) 10-2003-0072540
Oct. 17, 2003 (KR) 10-2003-0072541
Nov. 18, 2003 (KR) 10-2003-0081596

(51) Int. Cl. A61K 31/352

(52) U.S. Cl. 514/455

(57) ABSTRACT

The present invention relates to a composition for promoting collagen synthesis and a composition for external application to skin which includes the collagen synthesis promoting composition. The collagen synthesis promoting composition includes, as an effective component, at least one selected from the group consisting of compounds represented as: wherein R is hydrogen, a methoxy group, or a 3-methyl-2-butenyl group. The composition for external application to skin includes at least one collagen synthesis promoting component selected from the group consisting of compounds represented by above.

### TECHNICAL FIELD

The present invention relates to a composition for promoting the synthesis of collagen and a composition for external application to skin comprising the same, and more particularly, to a composition for promoting the synthesis of collagen which has an excellent collagen synthesis promoting effect and a composition for external application to skin that has an anti-wrinkle effect and an effect of healing wounds.

### BACKGROUND ART

Collagen is a major structural element of an extracellular matrix. It is a major structural protein generated in fibroblasts of skin and it exists in the extracellular matrix. It is a significant protein that accounts for about 30 percent of the total weight of human body protein and it has a firm triple helical structure. Collagen makes up most of the skin, tendon, bone, and organic material of teeth. Particularly, bone and dermis include a high level of collagen. In most of other body structural materials, it exists in the form of a fibrous inclusion body.

Collagen is also a sort of a relatively weak immunogen partially because it shuts off a potential antigenic determinant. The helical structure also gives collagen a resistance against decomposition of protein. Main functions of collagen are to give solidness to skin, give resistance and coherence to connective tissues, support coherence between cells, induce division and differentiation of cells (during the growth of an organism or wound healing), which was revealed by Van der Rest et al., in Ann NY Acad Sci., 1990. Collagen is known to be reduced by aging and by exposure to ultraviolet rays, and this is closely related to the formation of wrinkles on the skin, which was revealed by Arthur K. Balin et al., in Aging and the skin, in 1989. Also, collagen plays an important role in healing of wounds. Wounds can be healed quickly without a scar by promoting the synthesis of collagen in the wounded epidermis.

Conventionally, products obtained by mixing collagen with a composition for external application to skin, such as cosmetics and ointment are brought into the market to take advantage of the skin moisturizing effect and wound healing effect of collagen. However, since the collagen in those products has large molecules, it cannot be absorbed percutaneously just by being applied to the skin. Thus, the moisturizing effect and wound healing effect cannot be anticipated. Therefore, the products do not reveal the intrinsic skin improving function and the wound healing function.

In order to solve the problem, more attention is drawn to the development of a material that can promote the synthesis of collagen. Examples of conventionally known collagen synthesis promoting materials are vitamin C, retinoic acid, a transforming growth factor (TGF) (which was disclosed by Cardinale G. et al., in Adv. Enzymol., 41, p. 425, 1974), protein originated from animal placenta (JP8-231370), betulinic acid (JP8-208424), chlorella extract which promotes multiplication of fibroblasts (JP9-40523 and JP10-36283).

However, the above-mentioned materials cannot contribute to the substantial improvement in skin function or wound healing because they have insignificant effects or they have a limited quantity of use due to safety problems such as redness and irritation caused when they are applied to skin. Therefore, it is required to develop a composition for external application to skin which is safer to a human body and more effective than the conventional external skin application compositions.

### DISCLOSURE OF INVENTION

### Technical Problem

In order to resolve the problems described above, it is an aspect of the present invention to provide a composition for promoting synthesis of collagen with an excellent collagen synthesis promoting effect.

It is another aspect of the present invention to provide a composition for external application to skin which has an anti-wrinkle effect and a wound healing effect.

### Technical Solution

To achieve the aspects, the present invention provides a composition for promoting synthesis of collagen, the composition including at least one selected from the group consisting of compounds represented by the following Chemical Formula 1 as an effective compound.

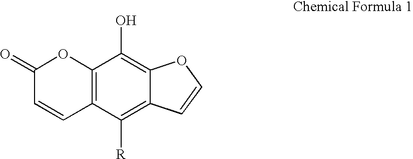

wherein R denotes hydrogen, a methoxy group, or a 3-methyl-2-buthenyl group.

The present invention further provides a composition for external application to skin which includes the collagen synthesis promoting composition which containing at least one selected from the group consisting of compounds represented by Chemical Formula 1.

### ADVANTAGEOUS EFFECTS

The collagen synthesis promoting composition of the present invention can promote the synthesis of collagen in fibroblasts of skin. The external skin application composition can improve the elasticity of the skin and reduce wrinkles effectively, and it also has excellent wound healing, anti-inflammatory, and antioxidative effects.

### DESCRIPTION OF DRAWINGS

FIG. 1 is a ¹H-Nuclear Magnetic Resonance (NMR) spectrum of xanthotoxol obtained in accordance with Example 1 of the present invention;

FIG. 2 is a ¹³C-NMR spectrum of xanthotoxol obtained in accordance with Example 1 of the present invention;

FIG. 3 is a mass spectrum of xanthotoxol obtained in accordance with Example 1 of the present invention;

FIG. 4 is a ¹H-NMR spectrum of 8-hydroxybergapten obtained in accordance with Example 2 of the present invention;

FIG. 5 is a ¹³C-NMR spectrum of 8-hydroxybergapten obtained in accordance with Example 2 of the present invention;

FIG. 6 is a mass spectrum of 8-hydroxybergapten obtained in accordance with Example 2 of the present invention;

FIG. 7 is a ¹H-NMR spectrum of prangenidin obtained in accordance with Example 3 of the present invention;

FIG. 8 is a ¹³C-NMR spectrum of prangenidin obtained in accordance with Example 3 of the present invention; and

FIG. 9 is a mass spectrum of prangenidin obtained in accordance with Example 3 of the present invention.

### BEST MODE

In the following detailed description, the following embodiments of the invention have been shown and described, simply by way of illustration of the best mode contemplated by the inventors of carrying out the invention. As will be realized, the invention is capable of modification in various respects, all without departing from the invention. Accordingly, the drawings and description are to be regarded as illustrative in nature, and not restrictive.

The inventor(s) of the present invention were developing a composition for promoting synthesis of collagen which can reduce wrinkles on the skin and heal wounds and a material having an excellent collagen synthesis promoting effect as an effective compound of a composition for external application to skin, when they discovered a strong collagen synthesis promoting effect from compounds represented by the following Chemical Formula 1 and completed the present invention.

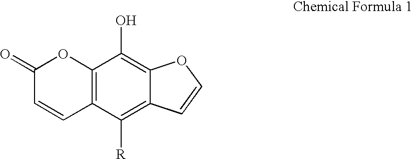

wherein R denotes hydrogen, a methoxy group, or a 3-methyl-2-buthenyl group.

The composition for promoting the synthesis of collagen, which will be referred to as a collagen synthesis promoting composition hereafter, includes as an effective compound at least one selected from the compounds represented by Chemical Formula 1.

Among the compounds represented by Chemical Formula 1, a compound where R is hydrogen is xanthotoxol, i.e., 8-hydroxypsoralen; a compound where R is a methoxy group is 8-hydroxybergapten, i.e., 5-benzofuranacrylic acid and 6,7-dihydroxy-4-methoxy-, delta-lactone; and a compound where R is 3-methyl-2-buthenyl group is prangenidin, i.e., 5-Benzofuranacrylic acid, 6,7-dihydroxy-4-(3-methyl-2-butenyl)-delta-lactone.

Since the compounds of Chemical Formula 1 are safe to a human body and have an effect of promoting the synthesis of collagen in fibroblasts of the skin, they are appropriate to be used for a collagen synthesis promoting composition. In addition, they have excellent effects of reducing wrinkles by improving the elasticity of the skin, as well as for healing wounds.

The compounds of Chemical Formula 1 mostly exist in the roots of umbelliferae plants and they can be acquired through various extracting methods. For example, the compounds can be obtained by chopping the roots of angelica dahurica (i.e., angelica dahuricae or angelica dahurica var. formosana), which is a plant of an umbelliferae order taxonomically, into pieces, putting the pieces into water, anhydrous or hydrous low molecular weight alcohol having 1 to 4 carbon atoms, ethylacetate, acetone, chloroform, or a combination thereof, heating the mixture for extraction, performing solvent fractionation, and then carrying out recrystallization.

The compounds of Chemical Formula 1 can be also acquired by first extracting imperatorin and cnidirin, which are major compounds of the angelica dahurica or angelica dahurica var. formosana, to improve the extraction efficiency of the effective compound and performing Claisen rearrangement thereon.

There is no restriction in the content of the compounds of Chemical Formula 1, which is included in the collagen synthesis promoting composition suggested in the present invention. Therefore, if a strong collagen synthesis promoting effect is needed, the collagen synthesis promoting composition can include the compounds up to 100 wt %. In short, the compound can be used freely within the effective content range, e.g., in the range of 0.00001 to 5 wt %.

The collagen synthesis promoting composition of the present invention can be applied to a human body in various forms. It can be dosed in an oral or parenteral method or it can be included in a liquid-type or solid-type carrier that can be admitted pharmacologically.

Also, the composition for external application to skin, which will be referred to as an external skin application composition hereafter, includes the compounds of Chemical Formula 1 as a collagen synthesis promoting composition. The external skin application composition is nonpoisonous to a human body and has excellent effects of restoring elasticity to skin, reducing wrinkles, and healing wounds.

The external skin application composition can include the compounds of Chemical Formula 1 in the range of 0.000001 wt % to 10 wt % with respect to the entire composition weight. Preferably, it includes the compounds of Chemical Formula 1 in the range of 0.001 wt % to 10 wt % and, most preferably, in the range of 0.1 wt % to 10 wt %. If the content of the compounds of Chemical Formula 1 is less than 0.000001 wt %, no remarkable effect is expected. If it exceeds 10 wt %, a further increase in the effect by the increase in the content does not appear.

The external skin application composition can be used as cosmetics for reducing wrinkles on the skin and/or as a drug for healing wounds.

The external skin application composition can be prepared in the forms of powder, gel, ointment, cream or liquid, and it can be used being mixed with one or more selected from the group consisting of an antibiotic, a coupling agent, a disintegrant, a diluent, a glossing agent, a stabilizer, a preservative, and an aromatic material.

Also, the external skin application composition can be prepared in forms of cream, foam, toilet water, cosmetic pack, skin softener, oil, foundation, makeup base, essence, soap, liquid rinse, a bathing additive, sunscreen cream, sun oil, and spray-type liquid products. It can be mixed with other general components used for preparing cosmetic compositions, such as oil, water, a surface active agent, a moisturizing agent, low molecular weight alcohol, a thickener, a chelate compound, pigment, an antiseptic, and an aromatic material.

### MODE FOR INVENTION

The following examples further illustrate the present invention in detail but they are not to be construed to limit the scope thereof.

### Example 1

### Extraction of Xanthotoxol

1-1. Extraction of Xanthotoxol Using Methanol

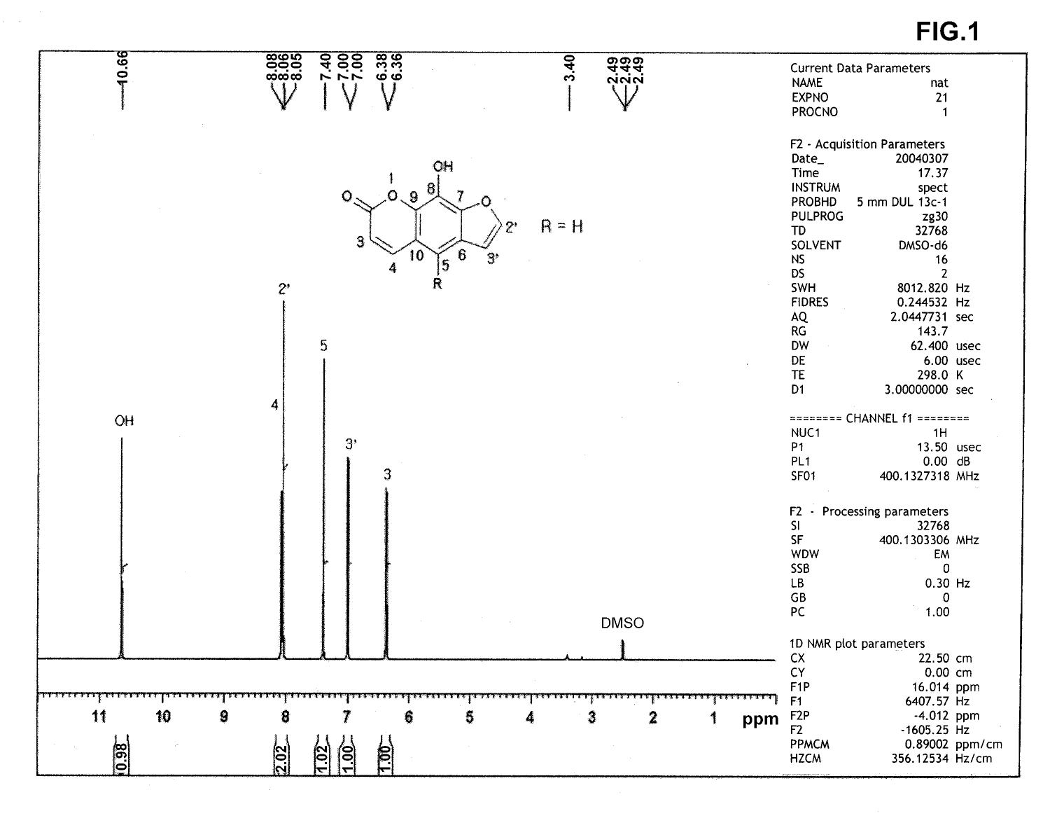

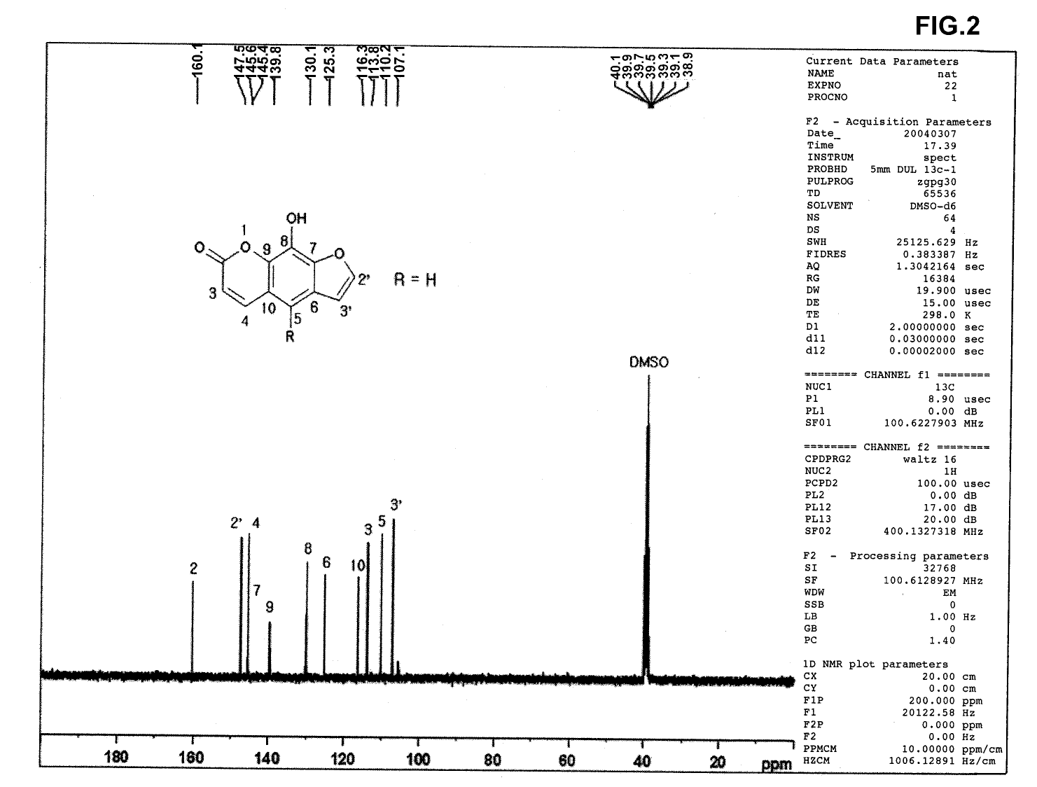

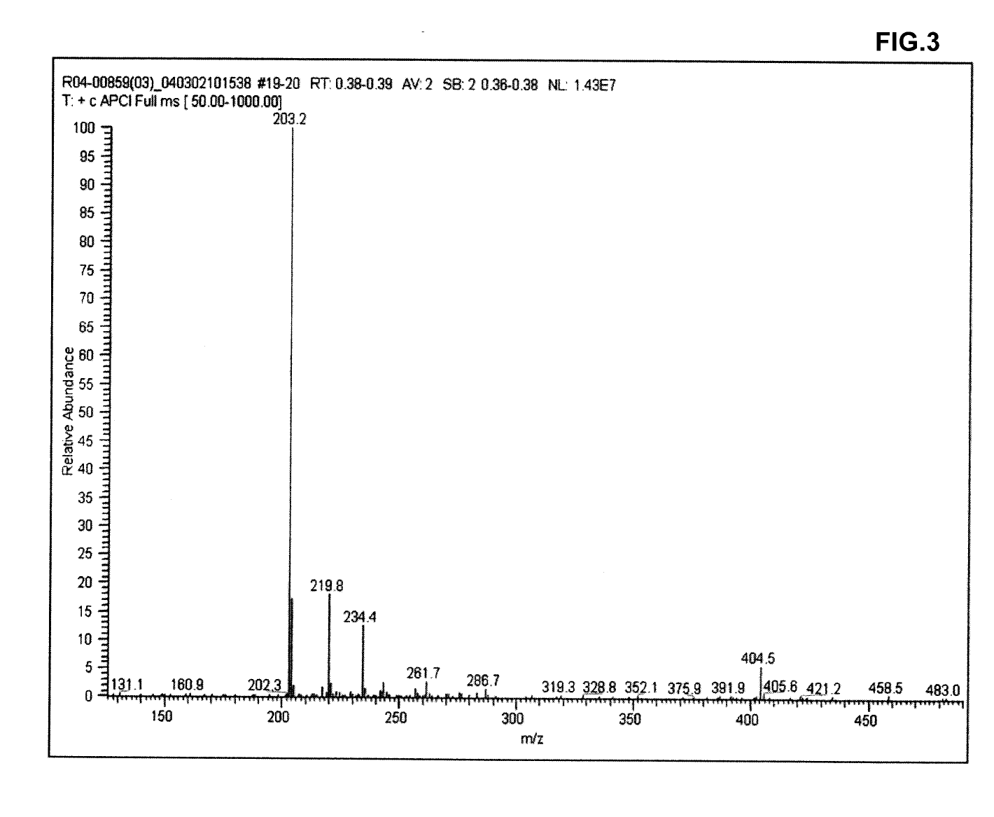

1 kg dried roots of angelica dahurica or angelica dahurica var. formosana were put into 10 l methanol and heated for extraction in an extractor with a Liebig condenser at 80° C. for 3 hours to thereby obtain 85 g of methanol extract. The methanol extract was removed from the hexane fraction through solvent fractionation. The obtained fraction was fractionated with chloroform three times to thereby obtain 9 g of a chloroform fraction. From the obtained chloroform fraction, 0.3 g of a fraction containing xanthotoxol was obtained through performing silica column chromatography several times. Xanthotoxol of the following Chemical Formula 2 was obtained by performing preparative High Performance Liquid Chromatography (prep-HPLC) and recrystallization on the xanthotoxol-containing fraction. The composition and contents of the above-obtained xanthotoxol were confirmed through Nuclear Magnetic Resonance (NMR) and Mass spectroscopy. FIGS. 1 and 2 show a ¹H-NMR spectrum and a ¹³C-NMR spectrum of xanthotoxol, respectively. In the drawings, the numbers over the peaks correspond to the numbers written in chemical formulas of FIGS. 1 and 2. FIG. 3 presents a mass spectrum of the xanthotoxol.

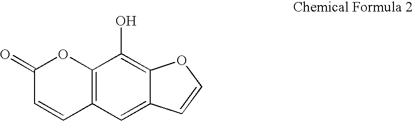

1-2. Extraction of Xanthotoxol Using Chloroform

1 kg dried roots of angelica dahurica or angelica dahurica var. formosana were put into 10 l chloroform and heated for extraction in an extractor with a Liebig condenser at 100° C. for 3 hours to thereby obtain 12 g of chloroform extract. The chloroform extract was dissolved in chloroform and solvent fractionation was performed thereon with an alkali aqueous solution, which was a 0.1M NaOH aqueous solution, to thereby obtain an alkali aqueous solution soluble fraction. The alkali aqueous solution soluble fraction was neutralized with HCl. Then, with 1 g of the chloroform fraction that was obtained by performing solvent fractionation with chloroform, prep-HPLC and recrystallization were carried out to thereby obtain xanthotoxol. The composition and contents of the above-obtained xanthotoxol were confirmed through the NMR and Mass spectroscopy. The content was 99.7 wt %.

1-3. Extraction and Chemical Deformation of Imperatorin and Cnidirin Using Methanol

1 kg of dried roots of angelica dahurica or angelica dahurica var. formosana were put into 10 l of methanol and heated for extraction in an extractor with a Liebig condenser at 80° C. for 3 hours, to thereby obtain 85 g of methanol extract. The methanol extract was fractionated with chloroform three times by performing the solvent fractionation to thereby obtain 11 g of a chloroform fraction. From the chloroform fraction, 7 g of a fraction containing imperatorin and cnidirin, which are major components of the angelica dahurica and angelica dahurica var. formosana, were obtained through silica column chromatography. Subsequently, the Claisen rearrangement reaction was carried out by adding 25 g of N,N-Diethylanilin to the fraction containing imperatorin and cnidirin and heating the mixture at 220° C. for one hour. The resultant was rinsed with a 5N HCl solution and then dissolved in chloroform and maintained in a refrigerator to induce precipitation. Xanthotoxol was obtained by performing the prep-HPLC and recrystallization on the above-obtained precipitation. The composition and contents of the above-obtained xanthotoxol were confirmed through the NMR and Mass spectroscopy. The content was 99.7 wt %.

### Example 2

### Extraction of 8-Hydroxybergapten

2-1. Extraction of 8-Hydroxybergapten Using Methanol

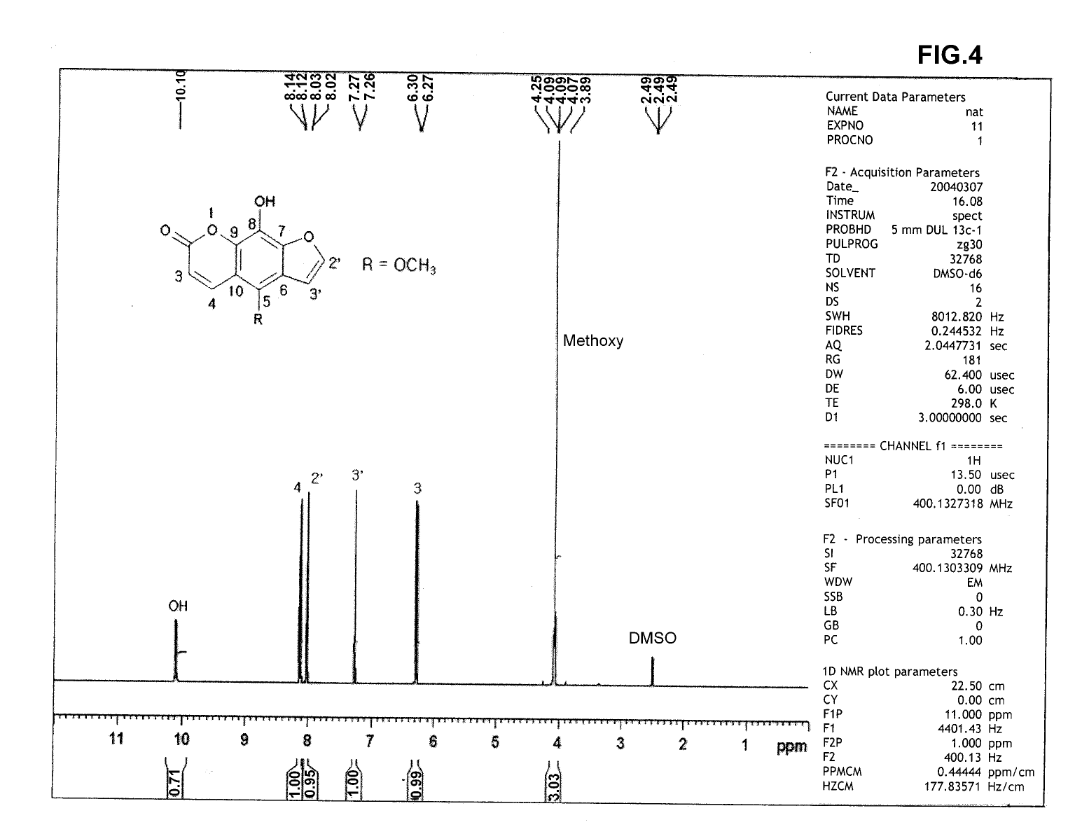

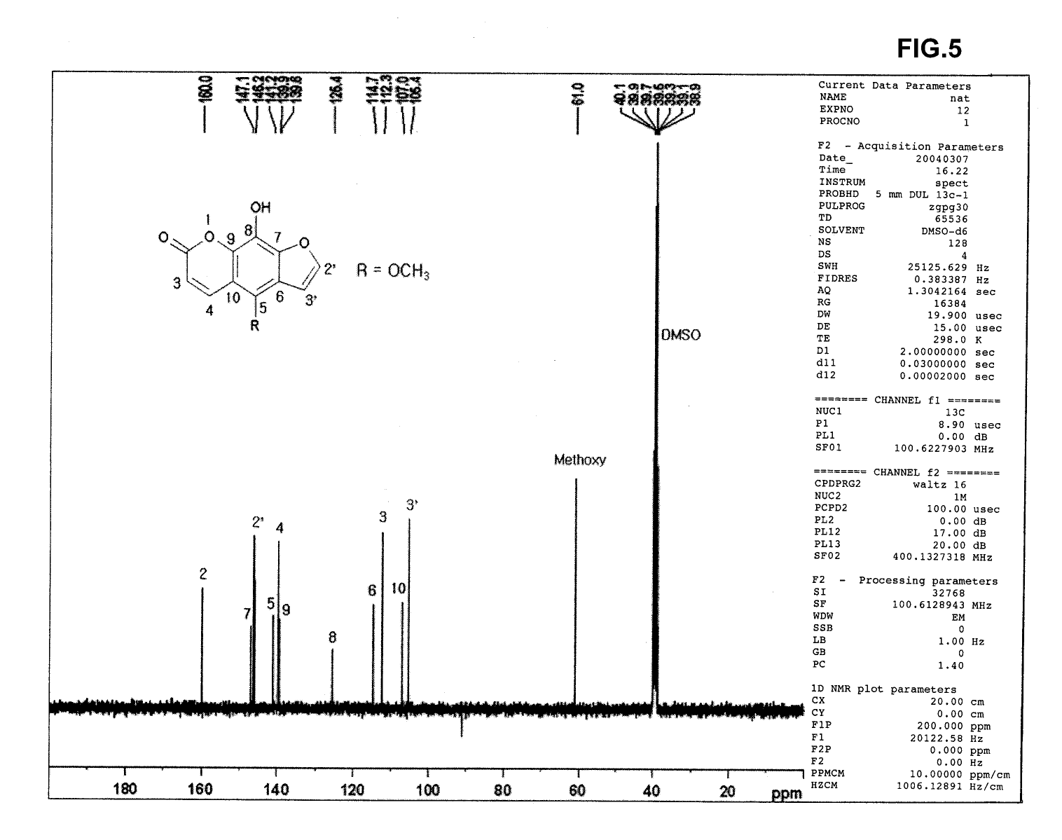

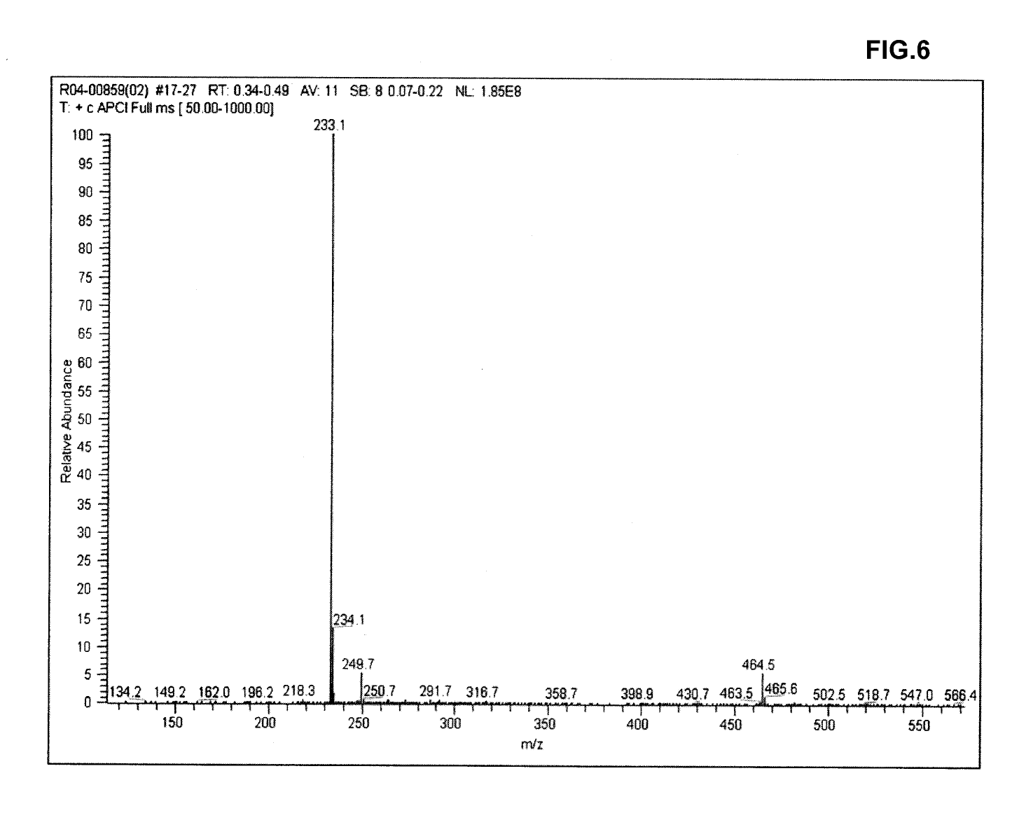

1 kg of dried roots of angelica dahurica or angelica dahurica var. formosana were put into 10 l of methanol and heated for extraction in an extractor with a Liebig condenser at 80° C. for 3 hours to thereby obtain 85 g of methanol extract. The methanol extract was removed from a hexane fraction through solvent fractionation. The obtained fraction was fractionated with chloroform three times to thereby obtain 9 g of a chloroform fraction. From the obtained chloroform fraction, 0.2 g of a fraction containing 8-hydroxybergapten was obtained through performing silica column chromatography several times. 8-hydroxybergapten of the following Chemical Formula 3 was obtained by performing prep-HPLC and recrystallization on the fraction containing 8-hydroxybergapten. The composition and contents of the above-obtained 8-hydroxybergapten were confirmed through NMR and Mass spectroscopy. The content was 99.7 wt %. FIGS. 4 and 5 show a ¹H-NMR spectrum and a ¹³C-NMR spectrum of the 8-hydroxybergapten, respectively. In the drawings, the numbers over the peaks correspond to the numbers written in the chemical formulas of FIGS. 4 and 5. FIG. 6 presents a mass spectrum of the 8-hydroxybergapten.

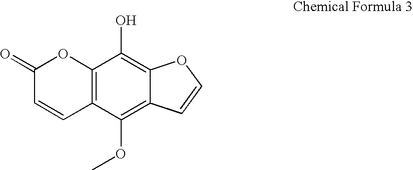

2-2. Extraction of 8-Hydroxybergapten Using Chloroform

1 kg of dried roots of angelica dahurica or angelica dahurica var. formosana were put into 10 l of chloroform and heated for extraction in an extractor with a Liebig condenser at 100° C. for 3 hours to thereby obtain 12 g of a chloroform extract. The chloroform extract was dissolved in chloroform and solvent fractionation was performed thereon with an alkali aqueous solution, which was a 0.1M NaOH aqueous solution, to thereby obtain an alkali aqueous solution soluble fraction. The alkali aqueous solution soluble fraction was neutralized with HCl. Then, with 1 g of the chloroform fraction that was obtained by performing solvent fractionation with chloroform, prep-HPLC and recrystallization were carried out to thereby obtain 8-hydroxybergapten. The composition and contents of the above-obtained 8-hydroxybergapten were confirmed through NMR and Mass spectroscopy. The content was 99.7 wt %.

2-3. Extraction and Chemical Deformation of Imperatorin and Cnidirin Using Methanol

1 kg of dried roots of angelica dahurica or angelica dahurica var. formosana were put into 10 l of methanol and heated for extraction in an extractor with a Liebig condenser at 80° C. for 3 hours to thereby obtain 85 g of a methanol extract. The methanol extract was fractionated with chloroform three times by performing the solvent fractionation to thereby obtain 11 g of a chloroform fraction. From the chloroform fraction, a 7 g fraction containing imperatorin and cnidirin, which are major components of the angelica dahurica and angelica dahurica var. formosana, were obtained through silica column chromatography. Subsequently, the Claisen rearrangement reaction was carried out by adding 25 g of N,N-Diethylanilin to the fraction containing imperatorin and cnidirin and heating the mixture at 220° C. for one hour. The resultant was rinsed with a 5N HCl solution and then dissolved in chloroform and maintained in a refrigerator to induce precipitation. Subsequently, 8-hydroxybergapten was obtained by performing prep-HPLC and recrystallization on the above-obtained precipitation. The composition and contents of the above-obtained 8-hydroxybergapten were confirmed through NMR and Mass spectroscopy. The content was 99.7 wt %.

### Example 3

### Extraction of Prangenidin

1-1. Extraction of Prangenidin Using Methanol

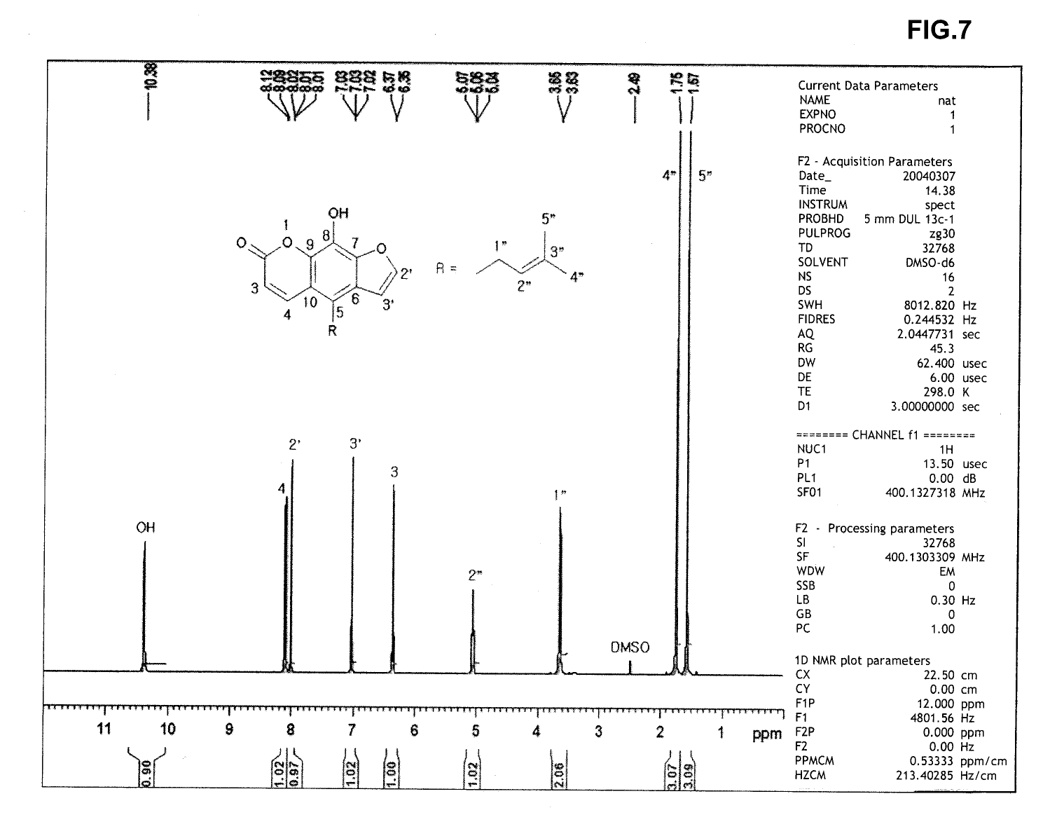

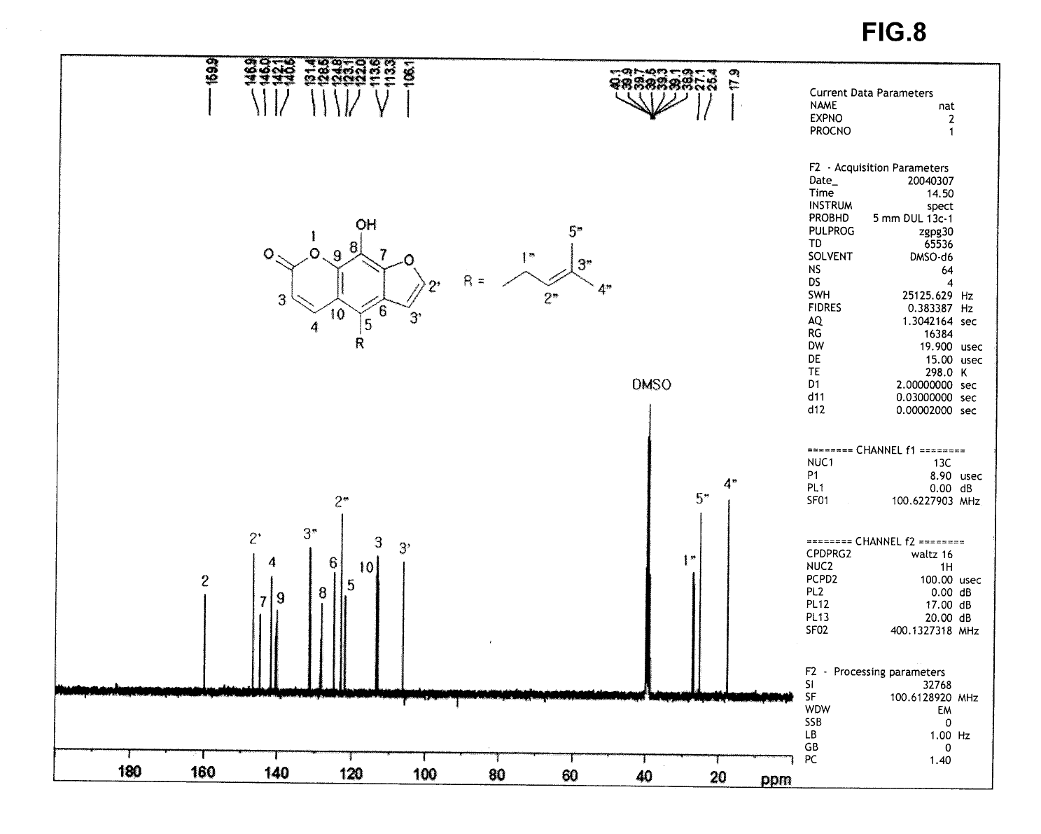

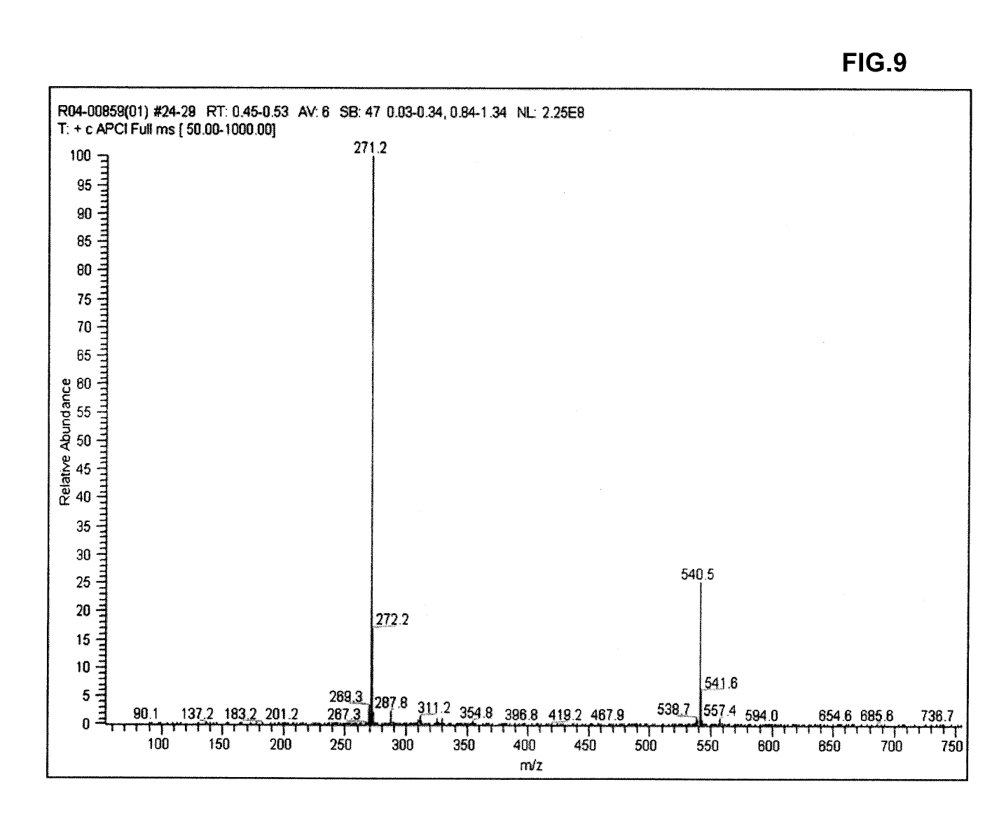

1 kg of dried roots of angelica dahurica or angelica dahurica var. formosana were put into 10 l of methanol and heated for extraction in an extractor with a Liebig condenser at 80° C. for 3 hours to thereby obtain 85 g of a methanol extract. The methanol extract was removed from the hexane fraction through solvent fractionation. The obtained fraction was fractionated with chloroform three times to thereby obtain 9 g of a chloroform fraction. From the obtained chloroform fraction, 0.3 g of a fraction containing prangenidin was obtained through performing silica column chromatography several times. Subsequently, prangenidin of the following Chemical Formula 4 was obtained by performing prep-HPLC and recrystallization on the prangenidin-containing fraction. The composition and contents of the above-obtained prangenidin were confirmed through NMR and Mass spectroscopy. FIGS. 7 and 8 show a ¹H-NMR spectrum and a ¹³C-NMR spectrum of prangenidin, respectively. In the drawings, the numbers over the peaks correspond to the numbers written in the chemical formulas of FIGS. 7 and 8. FIG. 9 presents a mass spectrum of the xanthotoxol.

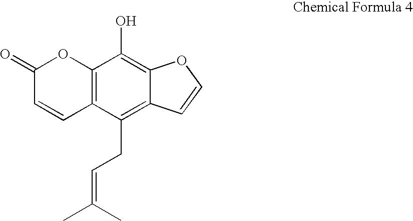

3-2. Extraction of Prangenidin Using Chloroform

1 kg of dried roots of angelica dahurica or angelica dahurica var. formosana were put into 10 l of chloroform and heated for extraction in an extractor with a Liebig condenser at 100° C. for 3 hours to thereby obtain 12 g of chloroform extract. The chloroform extract was dissolved in chloroform and solvent fractionation was performed thereon with an alkali aqueous solution, which was a 0.1M NaOH aqueous solution, to thereby obtain an alkali aqueous solution soluble fraction. The alkali aqueous solution soluble fraction was neutralized with HCl. Then, with 1 g of the chloroform fraction that was obtained by performing solvent fractionation with chloroform, prep-HPLC and recrystallization were carried out to thereby obtain prangenidin. The composition and contents of the above-obtained prangenidin were confirmed through NMR and Mass spectroscopy. The content was 99.7 wt %.

3-3. Extraction and Chemical Deformation of Imperatorin and Cnidirin Using Methanol

1 kg of dried roots of angelica dahurica or angelica dahurica var. formosana were put into 10 l of methanol and heated for extraction in an extractor with a Liebig condenser at 80° C. for 3 hours to thereby obtain 85 g of a methanol extract. The methanol extract was fractionated with chloroform three times by performing the solvent fractionation to thereby obtain 11 g of a chloroform fraction. From the chloroform fraction, 7 g of a fraction containing imperatorin and cnidirin, which are major components of the angelica dahurica and angelica dahurica var. formosana, were obtained through silica column chromatography. Subsequently, the Claisen rearrangement reaction was carried out by adding 25 g of N,N-Diethylanilin to the fraction containing imperatorin and cnidirin and heating the mixture at 220° C. for one hour. The resultant was rinsed with a 5N HCl solution and then dissolved in chloroform and maintained in a refrigerator to induce precipitation. Subsequently, prangenidin was obtained by performing prep-HPLC and recrystallization on the above-obtained precipitation. The composition and content of the above-obtained prangenidin were confirmed through NMR and Mass spectroscopy. The content was 99.7 wt %.

### Experimental Example 1

### Test for Collagen Synthesis Effect of Xanthotoxol

The collagen synthesis effect of Xanthotoxol was tested by adding Xanthotoxol to a culture broth for human fibroblasts. The quantity of the synthesized collagen was assayed by using a Procollagen Type I C-Peptide Enzyme ImmunoAssay (PICP EIA) Kit.

Xanthotoxol solution aliquots having final concentrations of 0.5 μg/ml, 1 μg/ml, 2 μg/ml, 5 μg/ml, and 10 μg/ml were added to a culture media for human fibroblasts, and the fibroblasts were cultured for one day. The culture broth of each concentration was taken out and the quantity of synthesized collagen was measured with a spectrophotometer at 450 nm by using the PICP EIA Kit.

To compare the effect of collagen synthesis, the human fibroblasts were cultured in a culture medium to which no extra element was added (i.e., a control group) and in a culture medium to which vitamin C having a final concentration of 52.8 μg/ml was added, and the quantity of synthesized collagen was measured with respect to the two samples with the same method as above.

The quantity of generated collagen was measured by using an ultraviolet ray (UV) spectrophotometer, and the increase rate of collagen generation was calculated in a relative ratio with respect to the collagen generation quantity of the control group. The result was as shown in Table 1.

TABLE 1
Collagen synthesis effect according to concentration (*
number of replications = 6)

| | | Quantity of | |
|---|---|---|---|
| | Concentration | Synthesized Collagen | Increase |
| Added Element | (μg/ml) | (Abs) | Rate (%) |
| Control group | — | 1.129 ± 0.053 | — |
| Xanthotoxol | 0.5 | 1.449 ± 0.039 | 128.36% |
| Xanthotoxol | 1.0 | 1.535 ± 0.078 | 135.94% |
| Xanthotoxol | 2.0 | 1.623 ± 0.084 | 143.75% |
| Xanthotoxol | 5.0 | 1.651 ± 0.130 | 146.24% |
| Xanthotoxol | 10.0 | 1.657 ± 0.112 | 146.81% |
| Vitamin C | 52.8 | 1.598 ± 0.145 | 141.57% |

As shown in Table 1, xanthotoxol has an excellent collagen synthesis effect with respect to the human fibroblasts, and the effect of using xanthotoxol was larger than the effect of using vitamin C, which is generally known to have a collagen synthesis effect.

### Experimental Example 2

### Test for Collagen Synthesis Effect of 8-hydroxybergapten

The quantity of synthesized collagen and the increase rate of collagen were measured with the same method as in Experimental Example 1, except that 8-hydroxybergapten was used instead of xanthotoxol. The result was as shown in Table 2.

TABLE 2
Collagen synthesis effect according to concentration
(* number of replications = 6)

| | | Quantity of | |
|---|---|---|---|
| | | Synthesized | Increase |
| | Concentration | Collagen | Rate |
| Added Element | (μg/ml) | (Abs) | (%) |
| Control group | — | 1.310 ± 0.072 | — |
| 8- | 0.5 | 1.590 ± 0.102 | 121.4 |
| hydroxybergapten | | | |
| 8- | 1.0 | 1.876 ± 0.097 | 143.2 |
| hydroxybergapten | | | |
| 8- | 2.0 | 1.949 ± 0.111 | 148.8 |
| hydroxybergapten | | | |
| 8- | 5.0 | 1.953 ± 0.132 | 149.1 |
| hydroxybergapten | | | |
| 8- | 10.0 | 2.008 ± 0.129 | 153.3 |
| hydroxybergapten | | | |
| Vitamin C | 52.8 | 1.757 ± 0.121 | 134.1 |

As shown in Table 2, 8-hydroxybergapten has an excellent collagen synthesis effect with respect to the human fibroblasts, and the effect of using 8-hydroxybergapten was larger than the effect of using vitamin C, which is generally known to have an ability to synthesize collagen.

### Experimental Example 3

### Test for Collagen Synthesis Effect of Prangenidin

The quantity of synthesized collagen and the increase rate of collagen were measured with the same method as in Experimental Example 1, except that prangenidin was used instead of xanthotoxol. The result was as shown in Table 3.

TABLE 3
Collagen synthesis effect according to concentration
(* number of replications = 6)

| | | Quantity of | |
|---|---|---|---|
| | | Synthesized | Increase |
| | Concentration | Collagen | Rate |
| Added Element | (μg/ml) | (Abs) | (%) |
| Control group | — | 1.330 ± 0.083 | — |
| Prangenidin | 0.5 | 1.537 ± 0.099 | 115.6 |
| Prangenidin | 1.0 | 1.778 ± 0.121 | 133.7 |
| Prangenidin | 2.0 | 1.801 ± 0.135 | 135.4 |
| Prangenidin | 5.0 | 1.813 ± 0.130 | 136.3 |
| Prangenidin | 10.0 | 1.820 ± 0.204 | 136.8 |
| Vitamin C | 52.8 | 1.739 ± 0.145 | 130.8 |

As shown in Table 3, prangenidin has an excellent collagen synthesis effect with respect to the human fibroblasts, and the effect of using prangenidin was larger than the effect of using vitamin C, which is generally known to have an ability to synthesize collagen.

### Experimental Example 4

### Test for Anti-Wrinkle Effect

The anti-wrinkle effects of xanthotoxol, 8-hydroxybergapten, and prangenidin were tested with respect to 6-week-old hairless mice having skin wrinkles caused by radiating light. Samples were prepared by extracting xanthotoxol, 8-hydroxybergapten, and prangenidin in accordance with Examples 1 through 3 and dissolving them in 1,3-butyleneglycol to thereby prepare 5 mg/ml sample solutions.

The skin wrinkles were caused by radiating light of 2 Minimum Erythema Dose (MED) to hairless mice with a solar simulator, three days a week for 10 weeks. Then, the improvement was assayed based on quality with respect to a control group of hairless mice treated with 1,3-butyleneglycol and test groups of hairless mice treated with the 5 mg/ml samples for 6 weeks.

The extent of wrinkle improvement was determined with the naked bare eye and by photographing the areas treated with the samples. The test groups and the control group were compared and the result was determined in three steps: no improvement; some improvement; and remarkable improvement. The result was as shown in Table 4.

TABLE 4
Wrinkle Improvement effect on the skin of hairless mice
(Population of each group = 10)

| | No | Some | Remarkable |
|---|---|---|---|
| Sample | Improvement | Improvement | Improvement |
| Control group | 9 | 1 | 0 |
| Xanthotoxol | 2 | 5 | 3 |
| 8-Hydroxybergapten | 0 | 4 | 6 |
| Prangenidin | 1 | 5 | 4 |

As shown in Table 4, xanthotoxol, 8-hydroxybergapten, and prangenidin have excellent anti-wrinkle effects.

### Experimental Example 5

### Test for Anti-Inflammatory Effect

The anti-inflammatory effect was evaluated in an ear swelling method by using 6-week-old hairless mice. The left ears of the hairless mice were used as controlled areas to be compared with the test areas, and the right ears of the hairless mice were used as the test areas. Prior to the test, both ears of all the hairless mice were measured three times. Sample solutions were prepared by extracting xanthotoxol, 8-hydroxybergapten, and prangenidin in accordance with Examples 2 and 3 and dissolving them in ethanol at 1 wt %, individually. Then, 20 μl/ear sample solutions were applied to the right ears of the hairless mice, and 20 μl/ear of ethanol was applied to the left ear thereof. An hour later, 2 mg/ear of arachidonic acid was applied to both ears of the hairless mice. After another hour had passed, the extent of ear edema was measured by using a micrometer, three times. Also, for comparison purposes, the anti-inflammatory effect of indomethacine, which is generally known as an anti-inflammatory agent, was measured with the same method.

The anti-inflammatory effect was a ratio of a thickness of an ear in the test area to the thickness of an ear in the controlled area. It was calculated based on the following Equation 1 and presented as a damage rate. The calculation result was as shown in Table 5.

Damage rate (%)=(thickness of ear in test area/thickness of ear in controlled area)×100 Equation 1

TABLE 5
Anti-inflammatory effect on hairless mice (Population of each
group = 3)

| | Concentration | Damage Rate |
|---|---|---|
| Sample | (wt %) | (%) |
| Xanthotoxol | 1 | 42 |
| 8-Hydroxybergapten | 1 | 45 |
| Prangenidin | 1 | 40 |
| Indomethacine | 1 | 51 |

As shown in Table 5, xanthotoxol, 8-hydroxybergapten, and prangenidin have an anti-inflammatory effect as good as indomethacine, which is known as an anti-inflammatory agent.

### Experimental Example 6

### Test for Wound Healing Effect by Collagen Synthesis

A wound healing effect was tested with respect to 5-week-old male rats by using a tension stiffness method that reflects the quality and quantity of restoration from wounds. The backs of the rats were shaved and wounded with a scalpel, and then the incision was sutured. Sample solutions were prepared by extracting 8-hydroxybergapten and prangenidin in accordance with Examples 2 and 3, and dissolving them in ethanol at 1 wt %, individually. The sample solutions were injected into the incisions at 0.5 ml/cm² once every day for 6 days. After 6 days, the rats were sacrificed and the skin around the wounds was removed. Three skin specimens of 1 cm-wide skin flakes crossing the incision line were prepared for each individual and their tensile strength (g/cm) was measured by using a rheometer. The measured tensile strength was used as an index of the strength of the generated collagen fiber. The tensile strength of the control group, to which only ethanol without the sample therein was applied, was taken as 100% and the increase in the tensile strength was measured as a relative strength thereto. The measurement result was as shown in the following Table 6.

TABLE 6
Wound healing effect on rats

| | Tensile Strength | Strength Rate |
|---|---|---|
| Sample | (g/cm) | (%) |
| Control Group | 550 | 100 |
| Xanthotoxol | 642 | 117 |
| 8-Hydroxybergapten | 680 | 124 |
| Prangenidin | 663 | 121 |

### Experimental Example 7

### Test for Antioxidative Effect

The antioxidative effects of 8-hydroxybergapten and prangenidin were measured by using 1,1-diphenyl-2-picrylhydrazyl (DPPH). The radical scavenging activity of each sample was calculated by measuring an optical density (O.D.) at 517 nm with a spectrophotometer. Sample solutions were prepared by extracting samples, i.e., 8-hydroxybergapten and prangenidin, in accordance with the methods of Examples 2 and 3 and mixing the samples in concentrations of 3 μg/ml, 5 μg/ml, 10 μg/ml, 12 μg/ml, 50 μg/ml, 80 μg/ml, and 100 μg/ml, individually. Then, experimental solutions were prepared by mixing the 500 μl aliquots of the tested sample solutions with 500 μl of a 1.5×10⁻⁴ M DPPH methanolic solution. The quantity of DPPH remaining in each experimental solution was measured 30 minutes after the preparation of experimental solutions by using the spectrophotometer at 517 nm. In order to subtract the inherent optical densities of the samples from the measurement values of the experimental solutions, the optical densities of the sample solutions of the same concentrations were also measured at 517 nm. A control solution was prepared by mixing 500 μl of a 1.5×10⁻⁴ M DPPH methanolic solution with 500 μl of a methanol solution and using the mixture 30 minutes after the preparation. The radical scavenging activity of each sample was calculated based on the following Equation 2.

$$Radical Scavenging Activity ( \% ) = 100 \times \frac{( \begin{matrix}O . D . of Control Solution - \\ O . D . of Experimental Solution\end{matrix} )}{O . D . of Control Solution} \qquad Equation 2$$

To determine the radical scavenging activity of each sample, a coefficient of correlation (R), which is a value in connection with the radical scavenging activity, was obtained, and an IC50, a value that scavenges DPPH by 50%, was calculated. Also, in order to compare the antioxidative effects, the same experiment was carried out with respect to vitamin C. The following Table 7 shows the calculated values.

TABLE 7
Antioxidative effect in the DPPH scavenging method

| | IC 50 | Coefficient of |
|---|---|---|
| Sample | (μg/ml) | Correlation (R) |
| Xanthotoxol | 24.0 | 0.9901 |
| 8-Hydroxybergapten | 20.3 | 0.9910 |
| Prangenidin | 21.6 | 0.9941 |
| Vitamin C | 8.43 | 0.9935 |

As shown in Table 7, 8-hydroxybergapten, and prangenidin suggested in the present invention have a superior antioxidative effect to vitamin C.

Hereafter, examples of the external skin application composition comprising the effective compound of the present invention will be presented.

### Examples 4 to 6 and Comparative Example 1

### Cosmetic Essence

Cosmetic essences having compositions shown in Table 8 were prepared.

TABLE 8

| | | | | Comparative |
|---|---|---|---|---|
| Composition (wt %) | Example 4 | Example 5 | Example 6 | Example 1 |
| Xanthotoxol | 1 | — | — | — |
| 8-Hydroxybergapten | — | 1 | — | — |
| Prangenidin | — | — | 1 | — |
| Propyleneglycol | 10.0 | 10.0 | 10.0 | 10.0 |
| Glycerine | 10.0 | 10.0 | 10.0 | 10.0 |
| Sodium hyaluronate | 5.0 | 5.0 | 5.0 | 5.0 |
| aqueous solution (1%) | | | | |
| Ethanol | 5.0 | 5.0 | 5.0 | 5.0 |
| Polyoxyethylene | 1.0 | 1.0 | 1.0 | 1.0 |
| hydrogenated castor | | | | |
| oil | | | | |
| Methyl | 0.1 | 0.1 | 0.1 | 0.1 |
| parahydroxybenzoate | | | | |
| Aromatic Material | 0.05 | 0.05 | 0.05 | 0.05 |
| Purified Water | to 100 | to 100 | to 100 | to 100 |

### Experimental Example 8

### Test for Anti-Wrinkle Effect of Cosmetic Essence Based on Panel Survey

Panel tests were performed with respect to the cosmetic essences prepared in accordance with Examples 4 to 6 and Comparative Example 1 to determine the anti-wrinkle effect of the cosmetic essences.

60 healthy females aged 35 to 50 were selected and divided into four groups, 15 subjects for each group. Then, the essences of Examples 4 through 6 and Comparative Example 1 were respectively applied to the faces of those in the first through fourth groups, once every day for three months.

After the three months had passed, the subjects were surveyed for the wrinkle declining effect in their skin, i.e., the anti-wrinkle effect.

The subjects were asked to evaluate the anti-wrinkle effect and improvement in the elasticity of their skin in the three steps of no improvement, some improvement, and remarkable improvement, compared with the state of their skin before the experiments. The results were as shown in Table 9.

TABLE 9
Anti-wrinkle effect of the examples of the present invention
(Based on survey)

| | No | Some | Remarkable |
|---|---|---|---|
| Sample | Improvement | Improvement | Improvement |
| Comparative | 9 | 5 | 1 |
| Example 1 | | | |
| Example 4 | 1 | 8 | 6 |
| Example 5 | — | 7 | 8 |
| Example 6 | — | 8 | 7 |

As shown in Table 9, the anti-wrinkle effect was excellent when the essences of Examples 4 to 6 of the present invention were applied.

### Experimental Example 9

### Test for Anti-Wrinkle Effect of Cosmetic Essence Based on Video Analysis

Panel tests were performed with respect to 60 healthy females aged 35 to 50 with the same method as in Experimental Example 8 to determine the anti-wrinkle effect of the essences prepared in accordance with Examples 4 to 6. Then, the anti-wrinkle effects of Examples 4 to 6 were analyzed through video analysis.

For the video analysis, replicas under eyes were extracted before and after the experiments (Xantopren, Bayer) and skin wrinkles were analyzed two-dimensionally through video analysis, and wrinkle densities were measured.

In the video analysis, a wrinkle decrease rate was an average ratio of wrinkle density after experiment to a wrinkle density before experiment. The measurement results were as shown in the following Table 10.

TABLE 10
Anti-wrinkle effect based on video analysis

| | Decrease Rate of |
|---|---|
| Sample | Wrinkle Density (%) |
| Comparative | 8 |
| Example 1 | |
| Example 4 | 42 |
| Example 5 | 47 |
| Example 6 | 42 |

As shown in Table 10, the wrinkle densities were decreased remarkably when the essences of Examples 4 to 6 of the present invention were applied, compared with the wrinkle density when the essence of Comparative Example 1 was used.

During the experiments, no skin irritation or adverse effects were found with the use of the essences of Examples 4 to 6.

### Examples 7 to 9 and Comparative Example 2

### Ointment for External Skin Application

Ointments for external skin application having compositions shown in the following Table 11 were prepared.

TABLE 11

| | | | | Comparative |
|---|---|---|---|---|
| Composition (wt %) | Example 7 | Example 8 | Example 9 | Example 2 |
| Xanthotoxol | 2 | — | — | — |
| 8-hydroxybergapten | — | 2 | — | — |
| Prangenidin | — | — | 2 | — |
| Diethyl sebacate | 8 | 8 | 8 | 8 |
| Spermaceti | 5 | 5 | 5 | 5 |
| Polyoxyethylene | 6 | 6 | 6 | 6 |
| Oleyletherphosphate | | | | |
| Sodium benzoate | 0.1 | 0.1 | 0.1 | 0.1 |
| Vaseline | to 100 | to 100 | to 100 | to 100 |

### Examples 10 to 12 and Comparative Example 3

### Cosmetic Cream

Cosmetic creams having compositions as shown in Table 12 were prepared.

TABLE 12

| | Example | Example | Example | Comparative |
|---|---|---|---|---|
| Composition (wt %) | 10 | 11 | 12 | Example 3 |
| Xanthotoxol | 0.5 | — | — | — |
| 8-hydroxybergapten | — | 0.5 | — | — |
| Prangenidin | — | — | 0.5 | — |
| Stearic acid | 15.0 | 15.0 | 15.0 | 15.0 |
| Cetanol | 1.0 | 1.0 | 1.0 | 1.0 |
| Potassium hydroxide | 0.7 | 0.7 | 0.7 | 0.7 |
| Glycerine | 5.0 | 5.0 | 5.0 | 5.0 |
| Propylene glycol | 3.0 | 3.0 | 3.0 | 3.0 |
| Antiseptic | 0.05 | 0.05 | 0.05 | 0.05 |
| Aromatic material | 0.05 | 0.05 | 0.05 | 0.05 |
| Purified water | to 100 | to 100 | to 100 | to 100 |

### Examples 13 to 15 and Comparative Example 4

### Skin Softener

Cosmetic creams having compositions as shown in Table 13 were prepared.

TABLE 13

| | Example | Example | Example | Comparative |
|---|---|---|---|---|
| Composition (wt %) | 13 | 14 | 15 | Example 4 |
| Xanthotoxol | 0.2 | — | — | — |
| 8-hydroxybergapten | — | 0.2 | — | — |
| Prangenidin | — | — | 0.2 | — |
| Ethanol | 10.0 | 10.0 | 10.0 | 10.0 |
| Polylauric acid | 1.0 | 1.0 | 1.0 | 1.0 |
| polyoxyethylene | | | | |
| sorbitan | | | | |
| Methyl | 0.2 | 0.2 | 0.2 | 0.2 |
| parahydroxybenzoate | | | | |
| Glycerine | 5.0 | 5.0 | 5.0 | 5.0 |
| 1,3-butylene glycol | 6.0 | 6.0 | 6.0 | 6.0 |
| Aromatic material | 0.05 | 0.05 | 0.05 | 0.05 |
| Pigment | 0.05 | 0.05 | 0.05 | 0.05 |
| Purified water | to 100 | to 100 | to 100 | to 100 |

### Examples 16 to 18 and Comparative Example 5

### Nutritive Lotion

Nutritive lotion having compositions as shown in Table 14 were prepared.

TABLE 14

| | Example | Example | Example | Comparative |
|---|---|---|---|---|
| Composition (wt %) | 16 | 17 | 18 | Example 5 |
| Xanthotoxol | 0.1 | — | — | — |
| 8-hydroxybergapten | — | 0.1 | — | — |
| Prangenidin | — | — | 0.1 | — |
| Vaseline | 2.0 | 2.0 | 2.0 | 2.0 |
| Sesquioleic acid | 0.8 | 0.8 | 0.8 | 0.8 |
| sorbitan | | | | |
| Polyoxyethylene | 1.2 | 1.2 | 1.2 | 1.2 |
| oleylethyl | | | | |
| Methyl | 0.1 | 0.1 | 0.1 | 0.1 |
| parahydroxybenzoate | | | | |
| Propylene glycol | 5.0 | 5.0 | 5.0 | 5.0 |
| Ethanol | 3.2 | 3.2 | 3.2 | 3.2 |
| Carboxyvinyl polymer | 18.0 | 18.0 | 18.0 | 18.0 |
| Potassium hydroxide | 0.1 | 0.1 | 0.1 | 0.1 |
| Pigment | 0.05 | 0.05 | 0.05 | 0.05 |
| Aromatic material | 0.05 | 0.05 | 0.05 | 0.05 |
| Purified water | to 100 | to 100 | to 100 | to 100 |

### Examples 19 to 21 and Comparative Example 6

### Cosmetic Pack

Cosmetic pack having compositions as shown in Table 15 were prepared.

TABLE 15

| | Example | Example | Example | Comparative |
|---|---|---|---|---|
| Composition (wt %) | 19 | 20 | 21 | Example 6 |
| Xanthotoxol | 0.2 | — | — | — |
| 8-hydroxybergapten | — | 0.2 | — | — |
| Prangenidin | — | — | 0.2 | — |
| Glycerine | 5.0 | 5.0 | 5.0 | 5.0 |
| Propylene glycol | 4.0 | 4.0 | 4.0 | 4.0 |
| Polyvinyl alcohol | 15.0 | 15.0 | 15.0 | 15.0 |
| Ethanol | 8.0 | 8.0 | 8.0 | 8.0 |
| Polyoxyethylene | 1.0 | 1.0 | 1.0 | 1.0 |
| oleylethyl | | | | |
| Methyl | 0.2 | 0.2 | 0.2 | 0.2 |
| parahydroxybenzoate | | | | |
| Pigment | 0.05 | 0.05 | 0.05 | 0.05 |
| Aromatic material | 0.05 | 0.05 | 0.05 | 0.05 |
| Purified water | to 100 | to 100 | to 100 | to 100 |

### Examples 22 and 23 and Comparative Example 7

### Skin Softener for Male

Skin softeners for male having compositions as shown in Table 16 were prepared.

TABLE 16

| | Example | Example | Example | Comparative |
|---|---|---|---|---|
| Composition (wt %) | 22 | 23 | 24 | Example 7 |
| Xanthotoxol | 0.1 | — | — | — |
| 8- | — | 0.1 | — | — |
| hydroxybergapten | | | | |
| Prangenidin | — | — | 0.1 | — |
| Ethanol | 55.0 | 55.0 | 55.0 | 55.0 |
| PEG-40 | 0.5 | 0.5 | 0.5 | 0.5 |
| Hydrogenated | | | | |
| castor oil | | | | |
| 1,3-butylene glycol | 1.0 | 1.0 | 1.0 | 1.0 |
| Glycereth-26 | 1.0 | 1.0 | 1.0 | 1.0 |
| Antiseptic | 0.05 | 0.05 | 0.05 | 0.05 |
| Aromatic material | 0.05 | 0.05 | 0.05 | 0.05 |
| Purified water | to 100 | to 100 | to 100 | to 100 |

As described above, the collagen synthesis promoting composition including at least one selected from the group consisting of xanthotoxol, 8-hydroxybergapten, and prangenidin as an effective compound has a quite strong collagen synthesis effect in the human fibroblasts. Also, the external skin application compositions including the collagen synthesis promoting composition have excellent anti-wrinkle effects, wound healing effects, anti-inflammatory effects, and antioxidative effects.

1. A method of promoting synthesis of collagen, by externally applying an effective component to skin, wherein the effective component is represented by: 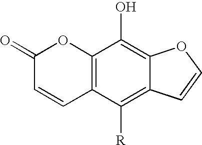
wherein R is a 3-methyl-2-butenyl group.

2. The method of claim 1 wherein the effective component represented by the chemical formula is applied in the concentration of 0.000001 wt % to 10 wt %.

3. The method of claim 1 wherein the effective component is externally applied to skin in a type selected from the group consisting of powder, gel, ointment, cream, and liquid,

4. The method of claim 1 wherein the effective component is externally applied to skin in a type selected from the group consisting of foam, toilet water, cosmetic pack, skin softener, lotion, foundation, makeup base, essence, soap, liquid rinse, a bathing additive, sunscreen cream, sun oil, and spray liquid.
